(19) United States
(12) Patent Application Publication
(10) Pub. No.: US 2022/0249513 A1
(43) Pub. Date: Aug. 11, 2022

(54) ACTIVITY OF GOLD-COMPLEXED COMPOUNDS AGAINST MYCOBACTERIUM TUBERCULOSIS AND MYCOBACTERIUM ABSCESSUS

(71) Applicants: UNIVERSITY OF CENTRAL FLORIDA RESEARCH FOUNDATION, INC., Orlando, FL (US); UNIVERSITY OF THE WITWATERSRAND, Johannesburg (ZA)

(72) Inventors: Kyle H. ROHDE, Orlando, FL (US); Orde Quentin MUNRO, Johannesburg (ZA)

(21) Appl. No.: 17/678,340

(22) Filed: Feb. 23, 2022

Related U.S. Application Data
(62) Division of application No. 16/624,535, filed on Dec. 19, 2019, now abandoned.
(60) Provisional application No. 62/525,013, filed on Jun. 26, 2017.

(51) Int. Cl. A61K 31/555; A61P 31/06

(52) U.S. Cl. CPC A61K 31/555; A61P 31/06

(57) ABSTRACT

Disclosed herein are gold compounds and methods of use thereof as antibacterials. Specifically exemplified are methods of treating a Mycobacterium infection by administering therapeutically effective amounts of the gold compounds.

### FIELD

The disclosure relates generally to antimicrobial compounds and more specifically to antimicrobial compounds comprising gold-complexed compounds.

### BACKGROUND

Mycobacterium tuberculosis (Mtb) and M. abscessus (Mab) are two important human pathogens belonging to the genus Mycobacteria. Mtb is a slow-growing mycobacterium that infects one-third of the human population and cause tuberculosis (TB) disease. It has been challenging to treat this disease with the emergence of drug-resistant strains. Presence of drug tolerant sub-populations poses an added challenge and necessitates long treatment times [1]. M. abscessus is a rapid growing non-tuberculous Mycobacterium that causes TB-like pulmonary infections as well as soft-tissue and wound infections [3]. It is increasingly encountered as the etiological agent of damaging pulmonary infections in patients with underlying lung conditions such as cystic fibrosis or bronchiectasis [2]. High level of intrinsic resistance of Mab to many chemotherapeutic agents, including front-line TB drugs, further exacerbates the problem. Clarithromycin, a cornerstone treatment for Mab infections is vulnerable to inducible drug resistance and high treatment failure rates [3, 4]. Both Mtb and Mab pulmonary infections require a long multidrug treatment therapy of over 6 months [5, 6]. Thus, there is an urgent need for potent novel therapeutics that are effective against drug resistant and dormant bacilli and potent enough to shorten treatment times.

Metal-containing complexes have a long history of use as chemotherapeutics. Silver and copper were used as antibacterial agents in ancient Greece [7]. The platinum-based drug cisplatin is widely used to treat genitourinary tumors [8], whereas Auranofin is a gold (I)-complexed compound licensed for rheumatoid arthritis condition [9, 10]. Other gold-containing compounds are used in the treatment of human diseases like asthma, cancer, HIV and malarial infections [7]. Metals, including gold, integrated into nanoparticles have proven to be potent antimicrobial agents even against multi-drug resistant bacteria [11-14]. The use of metal-based compounds against Mtb can be traced back to the bacteriologist Robert Koch who discovered that gold-cyanide can cause bacteriostatic inhibition of Mtb [7, 15]. Since that time, very few studies have reported activity of metal-containing compounds against mycobacteria [16, 17].

Inability to clearly differentiate Mtb and Mab pulmonary infections from each other owing to the very similar clinical symptoms and radiographic characteristics often leads to misdiagnosis of Mab infections as that of Mtb [18]. As a result, the treatment fails as most of the existing anti-TB drugs including the front-line anti-tubercular drugs such as rifampicin (RIF), isoniazid (INH), pyrazinamide, and ethambutol are not effective against Mab [19, 20]. There have been tremendous efforts to find TB drugs and unfortunately similar level of efforts are absent when it comes to Mab infections. Some efforts were aimed at repurposing TB drugs to find ones with activity against Mab with limited success [21-24] but it is believed that no study has evaluated or employed an approach to identify drugs active against both upfront.

### BRIEF DESCRIPTION OF DRAWINGS

These and other features, aspects, and advantages of the present disclosure will become better understood with reference to the following description and appended claims, and accompanying drawings where:

FIG. 1A is a schematic chemical structure of an exemplary gold-based antimicrobial compound, hereinafter referred to as “8”, “SA-8”, or “compound 8,” according to various embodiments;

FIG. 1B is a schematic chemical structure of an exemplary gold-based antimicrobial compound, hereinafter referred to as “10”, “SA-10”, or “compound 10,” according to various embodiments;

FIG. 1C is a schematic chemical structure of an exemplary gold-based antimicrobial compound, hereinafter referred to as “11”, “SA-11”, or “compound 11,” according to various embodiments;

FIG. 1D is a schematic chemical structure of an exemplary gold-based antimicrobial compound, hereinafter referred to as “14”, “SA-14”, or “compound 14,” according to various embodiments;

FIG. 1 E is a schematic chemical structure of an exemplary gold-based antimicrobial compound, hereinafter referred to as “15”, “SA-15”, or “compound 15,” according to various embodiments;

FIG. 2A is a chart of an example of dose dependent activity of gold (III) macrocycles and chelates, in concentrations ranging from 200 μM to 0.006 μM in 2-fold 16-point serial dilutions, against Mab, according to various embodiments, in which bacterial cultures were grown to log phase, diluted in 7H9 OADC and treated for 3 days after which the luminescence was read;

FIG. 2B is a chart of an example of dose dependent activity of gold (III) macrocycles and chelates, in concentrations ranging from 200 μM to 0.006 μM in 2-fold 16-point serial dilutions, against Mtb, according to various embodiments, in which bacterial cultures were grown to log phase, diluted in 7H9 OADC and treated for 5 days after which the luminescence was read;

FIG. 3A is a chart of an example of bactericidal activity of gold (III) macrocycle of structure 8 against Mtb, which was grown to log phase and diluted in 7H9 OADC, and then treated for 6 days, based on samples taken after each time point (0, 24, 72 and 144 h) from treated and untreated wells and plated on 7H10 OADC for colony enumeration, with compound concentrations ranging from MIC to 8-fold MIC, according to various embodiments;

FIG. 3B is a chart of an example of bactericidal activity of gold (III) macrocycle of structure 10 against Mtb, which was grown to log phase and diluted in 7H9 OADC, and then treated for 6 days, based on samples taken after each time point (0, 24, 72 and 144 h) from treated and untreated wells and plated on 7H10 OADC for colony enumeration, with compound concentrations ranging from MIC to 64-fold MIC, according to various embodiments;

FIG. 3C is a chart of an example of bactericidal activity of gold (III) macrocycle of structure 11 against Mtb, which was grown to log phase and diluted in 7H9 OADC, and then treated for 6 days, based on samples taken after each time point (0, 24, 72 and 144 h) from treated and untreated wells and plated on 7H10 OADC for colony enumeration, with compound concentrations ranging from MIC to 8-fold MIC, according to various embodiments;

FIG. 3D is a chart of an example of bactericidal activity of gold (III) chelate of structure 14 against Mtb, which was grown to log phase and diluted in 7H9 OADC, and then treated for 6 days, based on samples taken after each time point (0, 24, 72 and 144 h) from treated and untreated wells and plated on 7H10 OADC for colony enumeration, with compound concentrations ranging from MIC to 8-fold MIC, according to various embodiments;

FIG. 3E is a chart of an example of bactericidal activity of gold (III) chelate of structure 15 against Mtb, which was grown to log phase and diluted in 7H9 OADC, and then treated for 6 days, based on samples taken after each time point (0, 24, 72 and 144 h) from treated and untreated wells and plated on 7H10 OADC for colony enumeration, with compound concentrations ranging from MIC to 8-fold MIC, according to various embodiments;

FIG. 3F is a chart of an example of bactericidal activity of gold (III) chelate of structure 14 against Mab, which was grown to log phase and diluted in 7H9 OADC, and then treated for 3 days, based on samples taken after each time point (0, 24, 48 and 72 h) from treated and untreated wells and plated on 7H10 OADC for colony enumeration, with compound concentrations ranging from MIC to 8-fold MIC, according to various embodiments;

FIG. 4 is a chart of an example showing that drug resistant M. bovis BCG is sensitive to compound 14, according to various embodiments;

FIG. 5 is a chart of an example showing that compound 14 is active against clinical isolates of Mtb, according to various embodiments;

FIG. 6 is a chart of an example showing that compound 14 is active against dormant Mtb, according to various embodiments;

FIG. 7A is an example assay carried out with 10 ng EcTopo1, illustrating inhibition of bacterial topoisomerase 1A relaxation activity by compound 14, according to various embodiments, in which lane 1 is a control reaction without enzyme added; lane 2, DMSO control; lanes 3 to 10 are reactions with compound 10 at concentrations of 10, 5, 2.5, 1.25, 0.63, 0.31, 0.15, and 0.075 μM, respectively; and lanes 11 to 20 are reactions with compound 14 at concentrations of 160, 80, 40, 20, 10, 5, 2.5, 1.25, 0.62, and 0.31 μM, respectively;

FIG. 7B is an example assay carried out with 25 ng MtbTopo1, illustrating inhibition of bacterial topoisomerase 1A relaxation activity by compound 14, according to various embodiments, in which lane 1 is a control reaction without enzyme added; lane 2, DMSO control; lanes 3 to 10 are reactions with compound 10 at concentrations of 10, 5, 2.5, 1.25, 0.63, 0.31, 0.15, and 0.075 μM, respectively; and lanes 11 to 20 are reactions with compound 14 at concentrations of 160, 80, 40, 20, 10, 5, 2.5, 1.25, 0.62, and 0.31 μM, respectively;

FIG. 8A is an example assay of E. coli gyrase activity in the presence of compound 10, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 6 being reactions with compound 10 at concentrations of 10, 5, and 2.5 μM, respectively, according to various embodiments;

FIG. 8B is an example assay of E. coli gyrase activity in the presence of compound 14, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 9 being reactions with compound 14 at concentrations of 500, 250, 160, 80, 40, and 20 μM, respectively according to various embodiments;

FIG. 8C is an example assay of M. tuberculosis gyrase activity in the presence of compound 10, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 6, reactions with compound 10 at concentrations of 10, 5, and 2.5 μM, respectively according to various embodiments;

FIG. 8D is an example assay of M. tuberculosis gyrase activity in the presence of compound 14, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lane 4 to 15, reactions with compound 14 at concentrations of 1,000, 750, 500, 250, 160, 80, 40, 20, 10, 5, 2.5, and 1.25 μM, respectively, according to various embodiments;

FIG. 9 is an example assay illustrating human topoisomerase 1B (hTopo1) relaxation activity by compound 14, according to various embodiments, in which lane 1 is a control reaction without enzyme added; lane 2 is a DMSO control; lane 3 is a positive-control reaction with 200 μM camptothecin (CPT); and lanes 4 to 14 are reactions with compound 14 at concentrations of 160, 80, 40, 20, 10, 5, 2.5, 1.25, 0.62, 0.31, and 0.15 μM, respectively;

FIG. 10 is a schematic diagrammatic model of an example, illustrating topoisomerase inhibition by gold macrocycles, according to various embodiments.

It should be understood that the various embodiments are not limited to the arrangements and instrumentality shown in the drawings.

### DETAILED DESCRIPTION

Disclosed herein are synthesized gold (III) complexed macrocyclic and open chelate compounds for activity against both Mtb and Mab. It was shown that gold (III)-complexed compounds were active against both mycobacterial pathogens with bactericidal mode of action, with compound 14 having the greatest activity. The data also demonstrated activity of the compound embodiments against dormant Mtb bacilli as well indicating their potential to shorten the treatment times of Mtb infections.

### Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention generally are performed according to conventional methods well known in the art and as described in various general and more specific references, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Kandel, Schwartz, and Jessell, eds., Principles of Neural Science, 4th ed., McGraw-Hill/Appleton & Lange: New York, N.Y. (2000). Unless defined otherwise, all technical and scientific terms used herein have the same meaning as commonly understood by one of ordinary skill in the art.

As used herein and in the appended claims, the singular forms “a,” “an,” and “the” include plural references unless the content clearly dictates otherwise.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20% up or down (higher or lower).

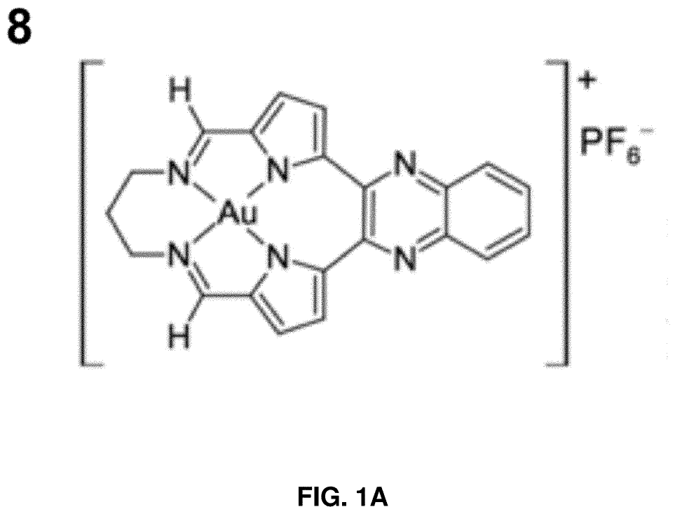

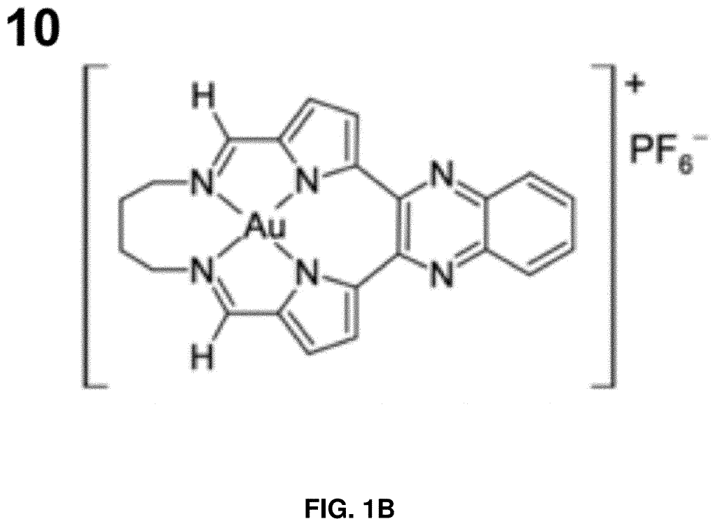

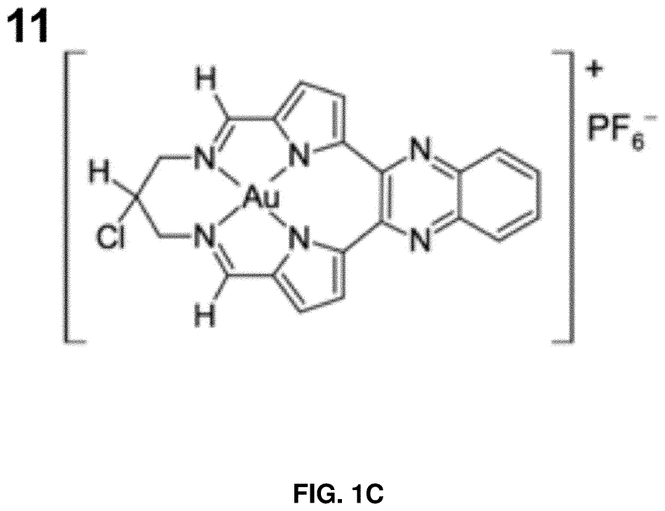

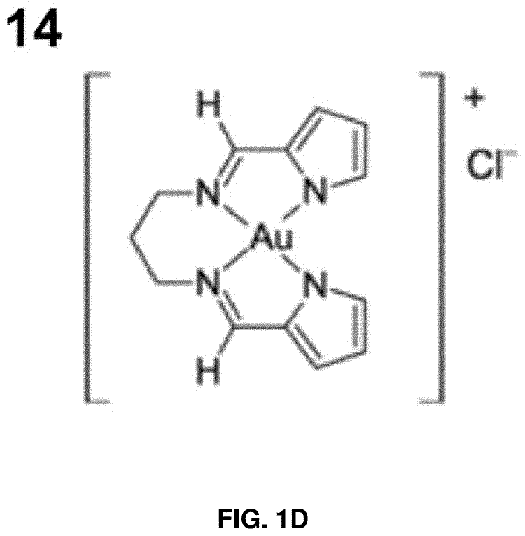

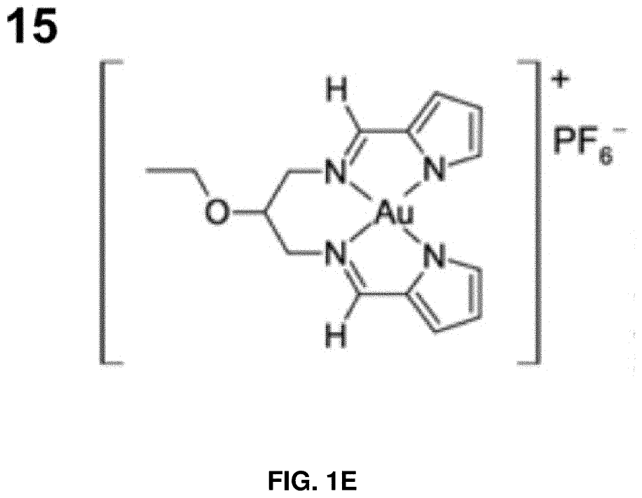

The term “enumerated agents” refers to gold-complexed compounds disclosed herein, including those, e.g., shown in FIG. 1.

The terms “subject,” “individual,” “host,” and “patient,” are used interchangeably herein to refer to an animal being treated with one or more enumerated agents as taught herein, including, but not limited to, simians, humans, avians, felines, canines, equines, rodents, bovines, porcines, ovines, caprines, mammalian farm animals, mammalian sport animals, and mammalian pets. A suitable subject for the invention can be any animal, preferably a human, that is suspected of having, has been diagnosed as having, or is at risk of developing a disease that can be ameliorated, treated or prevented by administration of one or more enumerated agents.

The term “administering” or “administration” as used herein means providing the agent to a subject using any of the various methods or delivery systems for administering agents or pharmaceutical compositions known to those skilled in the art. Modes of administering include, but are not limited to, oral administration, inhalation, nasal administration, topical, parenteral administration such as intravenous, subcutaneous, intramuscular or intraperitoneal injections, rectal administration by way of suppositories, transdermal administration, intraocular administration or administration by any route or method that delivers a therapeutically effective amount of the drug or composition to the cells or tissue to which it is targeted. Alternatively, routine experimentation will determine other acceptable routes of administration.

The term “co-administration”, “co-administered” or “co-administering” as used herein refers to the administration of an active agent (e.g. gold(III) complex compound) before, concurrently, or after the administration of another active agent (e.g. adjunct antibacterial agent) such that the biological effects of either agents overlap. The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects.

The term “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a disease, disorder, or condition or one or more symptoms or manifestations of a disease, disorder or condition.

A “therapeutically effective amount” refers to an amount which, when administered in a proper dosing regimen, is sufficient to reduce or ameliorate the severity, duration, or progression of the disorder being treated (e.g., microbe infection), prevent the advancement of the disorder being treated (e.g., microbe infection), or cause the regression of the disorder being treated (e.g., microbe infection). The full therapeutic effect does not necessarily occur by administration of one dose and may occur only after administration of a series of doses. Thus, a therapeutically effective amount may be administered in one or more administrations per day for successive days.

### Exemplary Gold(III) Macrocycle Compounds and Chelates

The gold (III) macrocycle compounds and chelates according to various embodiments may be compounds of the Formula (I),

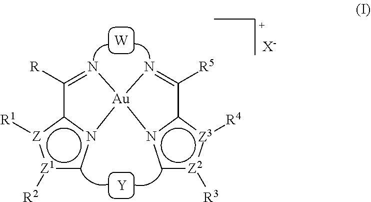

in which

W is independently selected from W¹, W², W³, W⁴, W⁵,

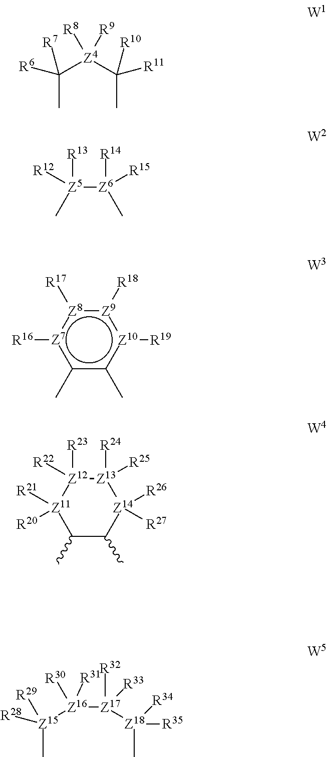

or W represents a pair of substituents independently selected from H, C₁-C₆ alkyl, Z⁵ or Z⁶ aryl or C₁-C₆ amide in which the amide is optionally part of a linking chain, and the Zⁿ-Z^{n′} bonds (n=4-17; n′=n+1) are optionally of any whole or partial bond order, Y is Y¹

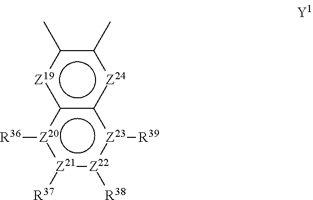

or Y represents a pair of substituents independently selected from H, C₁-C₆ alkyl, Z⁵ or Z⁶ aryl,
or Y is optionally a bridging structure that may comprise one or more C₁-C₆ amide, C₁-C₆ ether, or C₁-C₆ ester groups,
R—R³⁹ are independently selected from no substituent, a lone pair of electrons, H, halogen, C₅-C₆ aryl, C₁-C₁₂ alkyl, amine, C₁-C₆ alkylamine, C₁-C₆ amide, nitro, cyano, carboxyl, C₁-C₆ ester, phosphane, thiol, C₁-C₆thioether, OR⁴⁰, and suitable pairs of adjacent R groups (R—R³⁹) may optionally together form part of a C₅ or C₆ aryl ring, a Z⁵ or Z⁶ ring,
R⁴⁰ is independently selected from H, C₁-C₆ alkyl, Z⁵ or Z⁶ aryl, C₁-C₆ ester, poly(-C₂O—), amine, and C₁-C₆ alkylamine,
Z—Z²⁴ are independently selected from C, N, P, O, and S, and
X⁻ is a pharmaceutically acceptable anion.

The anion X⁻ may be selected from halide, hexafluorophosphate, nitrate, and triflate.

### Exemplary Embodiments

Various embodiments disclosed herein relate to methods of treating an object infected with microbes or at risk of infection comprising administering an effective amount of a composition, the composition comprising one or more of any of the gold (III) macrocycle compounds and chelates described herein, and optionally a pharmaceutically acceptable carrier.

According to one embodiment, provided is an antimicrobial composition comprising one or more of gold (III) macrocycle and chelate compounds and optionally a carrier. In a specific embodiment, the carrier is a pharmaceutically acceptable carrier. In another embodiment, the gold (III) macrocycle and chelate compounds comprise 8, 10, 11, 14 and 15.

Another embodiment disclosed herein is a method of treating an object infected with microbes or at risk of infection comprising administering an effective amount of a composition, the composition comprising one or more of compounds 8, 10, 11, 14 and 15, and optionally a carrier. In a specific embodiment, the carrier is a pharmaceutically acceptable carrier. In a specific embodiment, the object is a living subject infected with microbes or at risk of infection. In a more specific embodiment, the microbes comprise Mycobacterium spp. In an even more specific embodiment, the Mycobacterium spp comprises M. tuberculosis or M. abscessus, or both.

Another embodiment disclosed herein comprises a method of treating a subject infected with a Mycobacterium spp., the method comprising administering a therapeutically effective amount of a composition comprising one or more of compounds 8, 10, 11, 14 and 15. In a more specific embodiment, the composition comprises compound 14.

In addition to the gold compounds described above, Applicants refer to U.S. Pat. No. 9,346,832 ('832 patent) for further background on gold complexes and methods of making same. The entire contents of the '832 patent are incorporated herein by reference to the extent not inconsistent with the teachings herein. Furthermore, Akerman et al., Gold(III) Macrocycles: Nucleotide-Specific Unconventional Catalytic Inhibitors of Human Topoisomerase I, 2014, 136:5670-5682; and Wang, L.; Zhu, X. J.; Wong, W. Y.; Guo, J. P.; Wong, W. K.; Li, Z. Y. Dalton Trans. 2005, 3235-3240. provide background of gold macrocycles and synthesis thereof. One skilled in the art equipped with the teachings herein would be able to adapt the teachings of the preceding references to produce gold macrocycle compounds discussed herein, including 8, 10, 11, 14 and 15.

### Administration and Formulations

The compositions or pharmaceutical compositions described herein may be administered to the subject by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; as well as (d) administration topically; as deemed appropriate by those of skill in the art for bringing the active compound into contact with living tissue.

“Pharmaceutically acceptable carrier” is intended to include any and all solvents, binders, diluents, disintegrants, lubricants, dispersion media, coatings, antibacterial and antifungal agents, isotonic and absorption delaying agents, and the like, compatible with pharmaceutical administration. The use of such media and agents for pharmaceutically active substances is well known in the art. As long as any conventional media or agent is compatible with the active agent, such media can be used in the compositions of the invention and supplementary active agents or therapeutic agents can also be incorporated into the compositions. A pharmaceutical composition of the invention is formulated to be compatible with its intended route of administration.

Solutions or suspensions can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

For administration by inhalation, the composition can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.The exact formulation, route of administration and dosage for the gold compounds or pharmaceutical compositions containing such compounds can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). In one non-limiting embodiment, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are about 0.1% to about 500%, more preferably about 25% to about 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

One or more gold compounds described herein are generally administered in a therapeutically effective amount. Preferred doses range from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the subject treated and the particular mode of administration. Dosage unit forms will generally contain between about 1 mg to about 500 mg of an active ingredient. It will be understood, however, that the specific dose level for any particular subject may vary and will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of a compound to be administered may need to be optimized for each individual. The pharmaceutical compositions may contain the active ingredient in the range of about 0.1 to 2000 mg, preferably in the range of about 0.5 to 500 mg and most preferably between about 1 and 200 mg. The daily dose can be administered in one to four doses per day. Preferably, the daily dose is administered once per day.

### Adjunct Antibacterial Compounds

It is also contemplated herein that the gold(III) complexes described herein can be co-administered in combination with other antibacterial compounds (or adjunct compounds). Examples of adjunct antibacterial agents include, for example, an anti-tubercular agent useful for the treatment of tuberculosis in a mammal. Examples of such anti-tubercular agents include, rifampin, pyrazinamide, ethambutol, moxifloxacin, rifapentine, clofazimine, bedaquiline (TMC207), nitroimidazo-oxazine PA-824, delamanid (OPC-67683), oxazolidinone such as linezolid, tedizolid, radezolid, sutezolid (PNU-100480), and posizolid (AZD-5847), EMB analogue SQ109, a benzothiazinone, a dinitrobenzamide and an antiviral agent including an antiretroviral agent, or any TB agent being developed for the treatment of TB with a positive response in Phase IIa EBA trials, or any TB agent under development by the Global Alliance for Tuberculosis.

### EXAMPLES

### Materials and Methods

### Compounds

Stock solutions of Ciprofloxacin (CIP), Moxifloxacin (MOX), Rifampicin (RIF), isoniazid (INH) were prepared per the manufacturer's instructions. A total of 19 gold (III)-complexed macrocyclic and chelate compounds were synthesized and evaluated in this study for antimicrobial activity. These compounds were reconstituted by dissolving the powdered form in 100% DMSO at 10 mM concentration. The compounds were further diluted to appropriate assay concentrations in water.

### Bacterial Strains and Culture Conditions

Bacterial strains: Mtb CDC1551, 5 Mtb clinical isolates, Mab 390S, M. smegmatis, M. bovis and Escherichia coli used in this study are listed in Table 1.

TABLE 1
Strains and Plasmids

| Plasmid or | |
|---|---|
| strain | Genotype or phenotype |
| PLASMIDS | |
| pVVRG | Episomal plasmid expressing mCherry and Kan^{r} |
| pMV306hsp + | Integrative plasmid containing the luxCDABE operon, |
| LuxG13 | Kan^{r} |
| STRAINS | |
| CDC1551 | M. tuberculosis reference strain |
| Mtb-RG | M. tuberculosis CDC1551 expressing pVVRG |
| Mtb-lux | M. tuberculosis CDC1551 expressing pMV306hsp + |
| | LuxG13 |
| M. tuberculosis | M. tuberculosis clinical isolate 9532/03, Euro- |
| CI 1 | American lineage, Haarlem |
| M. tuberculosis | M. tuberculosis clinical isolate 2191/99, Euro- |
| CI 2 | American lineage, Uganda |
| M. tuberculosis | M. tuberculosis clinical isolate 1934/03, East Asian |
| CI 3 | lineage, Beijing |
| M. tuberculosis | M. tuberculosis clinical isolate 4850/03, Indo Oceanic |
| CI 4 | lineage, EAI |
| M. tuberculosis | M. tuberculosis clinical isolate 5468/02, West African |
| CI 5 | 2 lineage |
| M. abscessus | M. abscessus 390S, smooth colony phenotype |
| Mab-RG | M. abscessus 390S expressing PVVRG |
| Mab-lux | M. abscessus 390S expressing PMV306hsp + LuxG13 |
| M. abscessus | M. abscessus subsp. abscessus clinical isolates |
| CI 1 to 5 | |
| M. bovis BCG | Wild-type M. bovis |
| M. bovis BCG lux | Wild-type BCG expressing luxCDABE |
| M. bovis RIF^{r} | RIF-resistant BCG strain, rpoB-S531W, |
| lux | bioluminescent |
| M. bovis RIF^{r} | RIF- and FLQ-resistant BCG strain rpoB-S531L, |
| FLQ^{r} lux | gyrA-D94G, bioluminescent |

Mab strains are a kind gift from Dr. Thomas Byrd of The University of New Mexico [25]. Mtb and Mab strains were cultured in Middlebrook 7H9 supplemented with 0.05% Tween80 and 10% oleic acid/albumin/dextrose/catalase (OADC) and incubated at 37° C. and 5% CO₂ (normoxic) unless specified. Kanamycin 50 μg/ml (KAN), cycloheximide 100 μg/ml and amikacin 32 μg/ml (AMK) were added when appropriate.

### In Vitro Antimicrobial Susceptibility Assays

A total of 19 gold(III)-complexed compounds were examined for antimycobacterial activity using replicating mCherry reporter strains of Mtb and Mab (Mtb-RG and Mab-RG) [26]. For this initial screening against Mtb, gold-macrocycles and chelates were added at a final concentration of 5 μM to black solid-bottom 384-well screening plate (Corning) containing Mtb-RG culture of OD₆₀₀ 0.05 in a final total volume of 30 μl. Negative control (0.5% DMSO) and positive control (10 μM RIF) was also included. The screening plate was incubated at 37° C. and 5% CO₂ for 6 days. Evaluation of the compounds against Mab was carried out in a solid black 96-well plate with Mab-RG culture of OD 0.05. Compounds were added at 5 μM in a total volume of 100 μl per well. The screening plate was incubated for 3 days. Negative (0.5% DMSO) and the positive control (32 μg/ml AMK) was also included.

Fluorescence was measured in both Mtb and Mab screening plates using a Biotek Synergy plate reader at excitation/emission wavelengths of 485/575 nm. Percent inhibition was calculated using the formula [(negative control signal−sample signal)/negative control signal*100]. Z-factor was calculated as described [27].

### Exponential Growth Conditions

Gold(III)-complexed compounds were assayed for activity against replicating Mtb-RG and Mab-RG strains. Gold compounds at 5 μM (from a 5× stock) were added to a black solid-bottom 384-well plate (Corning) or to a solid-black 96-well plate (Corning) for Mtb and Mab, respectively. Mtb-RG culture at OD₆₀₀ 0.05 in a final total volume of 30 μl and Mab-RG culture was added to the screening plate containing the drugs in a total volume of 100 μl per well. Negative control (0.5% DMSO) and positive controls (10 μM RIF for Mtb, AMK 32 μg/ml for Mab) were included in the screening microtiter plate. After incubation at 37° C. and 5% CO₂ for various times (Mtb-6d, Mab-3d), mCherry fluorescence was measured using a Biotek Synergy plate reader at excitation/emission wavelengths of 485/575 nm. Percent inhibition was calculated using the formula {(negative control signal−sample signal)/negative control signal*100). Z-factor was calculated as described [27]( )

Activity of gold(III) chelate compound 14 was also evaluated against 5 clinical strains of Mtb as well against M. bovis, M. smegmatis and a non-mycobacterial Escherichia coli strain. M. bovis is tagged with luciferase and M.smegmatis with fluorescent mCherry reporter. Plasmid pVVRG was introduced into M. smegmatis to construct a Msmeg-RG strain. For assays involving Mtb clinical strains and E. coli, resazurin dye was added and fluorescence was measured at λ Ex/Em 530/590. Growth was reflected by the resazurin color change [25].

### Dormant Conditions

Dormant conditions were conducted as was a previously described multi-stress dormancy model (MSD) [26] to evaluate activity of gold-complexed compounds against dormant Mtb. Briefly, Mtb-lux grown in multiple-stress media for 9 days was added to the 384-well white solid bottom plates at an OD 0.1. Gold(III)-macrocyclic and chelate compounds, 10, 11, 14, 15 and 18 were added at a final concentration of 5 μM to the screening plate in a total volume of 30 μl. 0.5% DMSO, RIF and INH controls were also included. The plate was incubated under hypoxia conditions (10% CO₂ and 5% O₂) for 5 days and samples were serially diluted and plated on 7H10 OADC for CFU enumeration after 3 weeks of incubation at 37° C.

In parallel, we confirmed the tolerance of 9-day dormant Mtb-lux culture to rifampicin and isoniazid by comparing it to the replicating Mtb culture grown under normoxic conditions. Briefly, Mtb-lux grown in replicating Complete Dubos medium and multiple stress media was added to two separate 384-well white solid bottom plates with 8-point dilutions of Rif (0.5-0.003 μM) and Inh (1-0.015 μM). Negative control (0.5% DMSO) was also included in both the plates. Plates were appropriately incubated under normoxic (Complete Dubos medium, 37° C., 5% CO₂) and hypoxic conditions for 5 days. Aliquots were diluted and plated on 7H10 OADC for CFU enumeration.

### MIC Assay

MIC of 5 compounds (8, 10, 11, 14, and 15) was determined using bioluminescent strains of Mab and Mtb in solid white 384-well microtiter plates (Corning). A 2-fold serial dilution series of the compounds (200-0.006 μM) was carried out in 384-well white solid bottom plates while maintaining the final 2% DMSO concentration in each dilution. Log phase cultures (OD600=0.4-0.8) of Mab 390S-lux and Mtb-lux were diluted to 0.01 before addition to the wells in a total volume of 30 μl. Untreated DMSO controls were also included. Similarly, MIC of compound 14 was determined under dormant conditions as well using the 9-day hypoxia adapted CDC1551-lux culture, the details of which are described earlier. The plates were incubated at 37° C., 5% CO₂ until luminescence was read at 0, 24, 48 and 72 h using a plate reader. The data expressed as percent growth was fitted by a modified Gompertz model [28] and the dose-response curve was generated using GraphPad Prism 7.0. MIC is defined as the lowest drug concentration at which more than 99% of bacterial growth is inhibited as compared to the untreated control.

### Cytotoxicity Assay

Cytotoxicity was assessed on J774A.1 (ATCC® TIB67™) macrophage cell line. Macrophages were cultured in Dulbecco's Modified Eagle Medium (DMEM, GIBCO) supplemented with 10% heat inactivated fetal calf serum (Atlanta Biologicals), 1 mM sodium pyruvate (CellGro), 2 mM L-glutamine (CORNING) and 1% PenStrep (100 U/mL Penicillin, 100 mg/mL Streptomycin, GIBCO). Macrophages were seeded in a 384-well black transparent bottom plate (Corning) at 2.5×10⁴ cells/well. After macrophages were allowed to adhere for 4-6 h, a two-fold dilution series of the compounds (200-0.006 μM) and controls (2% Triton X and 0.5% DMSO) were added to the wells. The plate was incubated for 24 h and then 1/10^{th} volume of resazurin dye was added. Fluorescence was measured following 4 h incubation with the dye at an excitation of 560 nm and emission at 590 nm with cell viability indicated by conversion of resazurin to fluorescent product resofurin. Viability is determined as percent growth calculated relative to the DMSO control.

### Time-Kill Kinetic Assay

To examine whether bacterial killing is concentration and/or time-dependent, a time-kill kinetic study was carried out with 8, 10, 11, 14, and 15. For Mtb, CDC1551 cultures diluted to an OD of 0.01 in 7H9 were added to a solid-white 384-well plate containing compounds at final concentrations of 0, 1×, 4× and 8× MIC in a total volume of 30 μI. DMSO control was also included. The plate was incubated for 6 days for Mtb. At every time point-0, 24, 48 and 72 h post-inoculation, an aliquot was taken, serially diluted and 50 μl plated onto 7H10 quad-plates supplemented with OADC. Colonies were counted after 3-4 weeks of incubation at 37° C. and CFU/ml was calculated. Similarly, a time-kill assay for compound 14 was performed with Mab 390S culture with some changes. The plate was incubated for 3 days and colonies were counted after 5 days of incubation following plating. A 1-2 log₁₀ decrease in CFU/ml is considered bactericidal. Luminescence was also read at every time point before taking an aliquot for serial dilution.

### In Vitro Activity of Compound 14 Against Clinical Strains

Gold(III) chelate compound 14 was also evaluated against 5 clinical strains of Mtb from 5 different phylogenetic lineages (Table 1) at 5 μM concentration in a total volume of 30 μl in black 384-well solid bottom plates. The screening plate was incubated for 5 days at 37° C., thereafter resazurin dye was added at 1/10^{th} of the total volume and incubated for 24 h. Fluorescence was measured at λ Ex/Em=530/590 [29].

### Activity of Compound 14 Against Dormant Mtb

Employed was a previously described multi-stress dormancy model (MSD) [30] to evaluate activity of gold-complexed compound 14 against dormant Mtb. Briefly, Mtb-lux was grown in multiple-stress media (10% Complete Dubos at pH 5.0 containing 0.018% tyloxapol and no glycerol) and adapted for 9 days in a hypoxia chamber. This culture was added to the 384-well white solid bottom plates at an OD of 0.1. Gold(III chelate compound 14 was added at a final concentrations of 1×, 2×, 4× and 8× MIC determined under replicating conditions in a total volume of 30 μl. DMSO (0.5%), RIF and INH controls were also included. The plate was incubated under hypoxic conditions (37° C., 10% CO₂ and 5% O₂) for 5 days and samples were serially diluted and plated on 7H10 OADC for CFU enumeration after 3 weeks of incubation at 37° C.

In parallel, it was confirmed that the tolerance of 9-day dormant Mtb-lux culture to RIF and INH by comparing it to the Mtb-lux culture grown under normoxic conditions (37° C., 5% CO₂). Briefly, Mtb-lux grown in multiple stress media and in Complete Dubos medium supplemented with 10% Dubos-medium-albumin-supplement was added to two separate 384-well white solid bottom plates with 8-point dilutions of RIF (0.5-0.003 μM) and INH (1-0.015 μM). Negative control (0.5% DMSO) was also included in both the plates. Plates were appropriately incubated under hypoxic and normoxic conditions for 5 days. Aliquots were diluted and plated on 7H10 OADC for CFU enumeration. A shift in MIC of the culture grown in hypoxia condition as compared to the normoxic culture indicates the dormant nature of Mtb.

### BCG Drug Resistant Strains Construction

M. bovis (BCG) strains resistant to rifampicin and ciprofloxacin were obtained by selecting for spontaneous mutations in rpoB and gyrase genes, respectively. Briefly, BCG grown on 7H9/OADC media was plated onto 7H10 agar plates containing either 1 μg/ml RIF or 2 μg/ml CIP. Spontaneous resistant colonies were grown, PCR amplified for rpoB and gyrA genes and sequence confirmed. The rpoB primers-rpoB_sym200_F: 5′ GTCGCCGCGATCAAGGAGTT 3′ and rpoB_sym200_R: 5′ CCCTCAGGGGTTTCGATCGGG 3′ and gyrA primers-gyrA.SNP.PCR-F: 5′ ATTGCCGTTCCACGGATC 3′ and gyrA.SNP.PCR_R: 5′ GGGCGATATCGACGGTCT 3′ were used. A dual RIF and fluoroquinolone resistant mutant strain was generated in the RIFR strain background by the same process. These BCG drug resistant strains-BCG DR-1, BCG DR-2 and BCG DR-3 and the wild-type BCG were electroporated with plasmid pMV306hsp+LuxG13 [31]. The plasmid is a gift from Brian Robertson and Siouxsie Wiles (Addgene #26159). Transformants were selected on 7H10 KAN plates. To confirm their drug resistant phenotype, a dose-response curve analysis for RIF (12-0.0004 μM), CIP (64-0.002 μg/ml), and gold chelate 14 (200- 0.006 μM) was performed with these autoluminescent BCG drug resistant strains along with the wild-type BCG control as described earlier.

### Results

### Gold Compounds Effectively Inhibit Replicating Mtb and Mab

Initially 19 gold(III) macrocycles and chelates (as well as some vanadium(IV) chelates) were screened at a single concentration (5 μM) for 3 and 5 days against Mab-RG and Mtb-RG, respectively. Five compounds inhibited Mtb and four inhibited Mab above the defined threshold levels (>50% inhibition relative to untreated controls) (data not shown). Compounds 10 and 11, 14, 15 were identified which inhibited both mycobacterial species and one compounds (8) showed >50% inhibition of only Mtb-RG.

A luminescent readout (Mtb-Lux and Mab-Lux) was chosen for secondary assays to further characterize the activity of the hit compounds. The MICs of 10 were the lowest (Mtb—0.12 μM, Mab—5.4 μM), followed by 14 (Mtb—0.98 μM, Mab—11.9 μM). The SI (considering Mtb MIC) of these two compounds were 172 and 42, respectively. The chemical structures the five most active scaffolds are shown in FIG. 1.

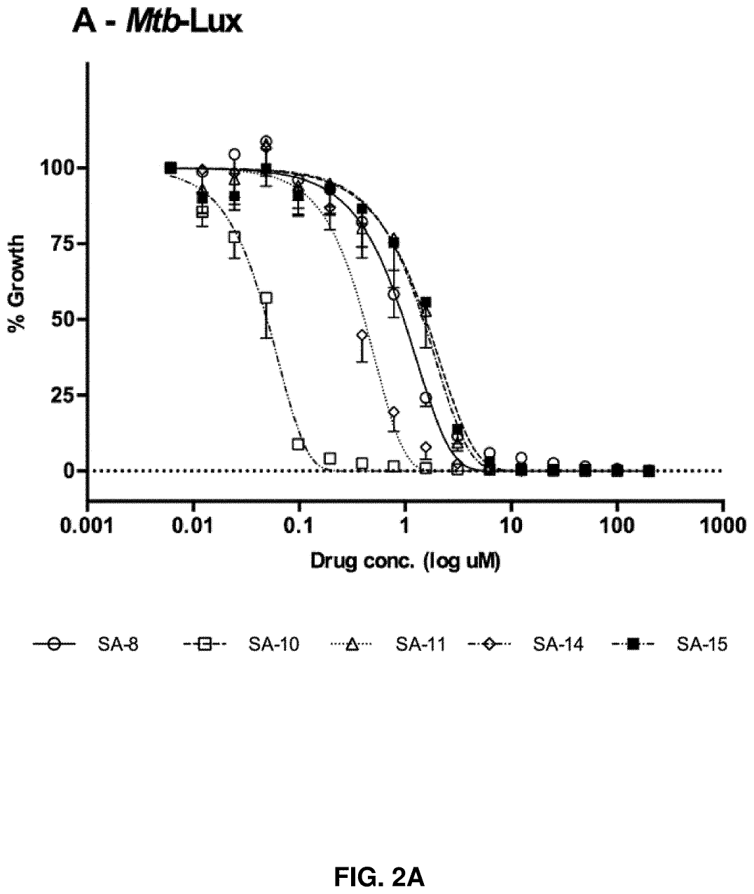

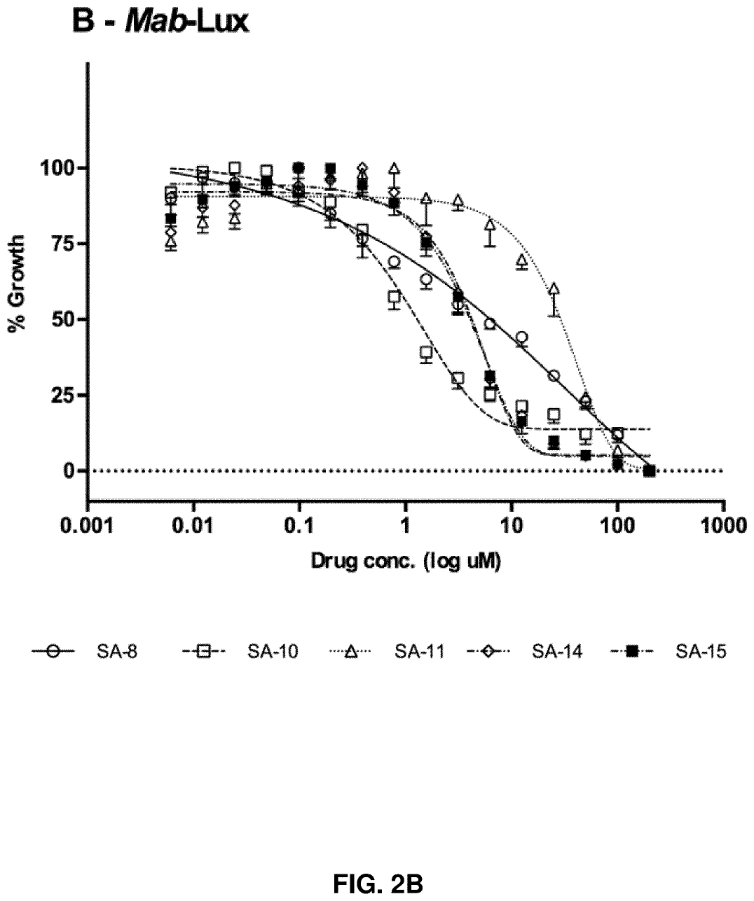

FIG. 2A is a chart of an example of dose dependent activity of gold(III) macrocycles and chelates, in concentrations ranging from 200 μM to 0.006 μM in 2-fold 16-point serial dilutions, against Mab, according to various embodiments, in which bacterial cultures were grown to log phase, diluted in 7H9 OADC and treated for 3 days after which the luminescence was read. FIG. 2B is a chart of an example of dose dependent activity of gold(III) macrocycles and chelates, in concentrations ranging from 200 μM to 0.006 μM in 2-fold 16-point serial dilutions, against Mtb, according to various embodiments, in which bacterial cultures were grown to log phase, diluted in 7H9 OADC and treated for 5 days after which the luminescence was read. Comparison of dose-response curves for the dual-active compounds (FIG. 2) revealed that all hit compounds were less potent against Mab than Mtb. This could be due to a higher affinity of the molecules for a Mtb target, or due to differences in the permeability of the cell walls of these two mycobacterial pathogens. The IC₅₀ of hits was determined against J774 macrophages, and IC₅₀ values for six compounds ranged from 43-109 μM which yielded selectivity indices >10 (IC₅₀/Mtb MIC). The complete antimicrobial and cytotoxicity profiles are shown in Table 2.

TABLE 2
Activity Profiles of 6 Active Scaffolds

| | MIC | | IC₅₀ | SI | |
|---|---|---|---|---|---|
| Compound | Mtb | Mab | J774 | Mtb MIC | Mab MIC |
| 8 | 2.72 | 849.41 | 48.48 | 17.82 | 0.057 |
| 10 | 0.12 | 5.44 | 85.82 | 715.16 | 15.77 |
| 11 | 4.06 | 91.72 | 108.5 | 26.72 | 1.18 |
| 14 | 0.98 | 11.98 | 42.3 | 43.16 | 3.53 |
| 15 | 4.61 | 13.05 | 48.89 | 10.6 | 3.74 |
| 18 | 9.75 | 91.93 | 102.9 | 10.55 | 1.11 |

### Gold Compounds Exhibit Diverse Modes of Action

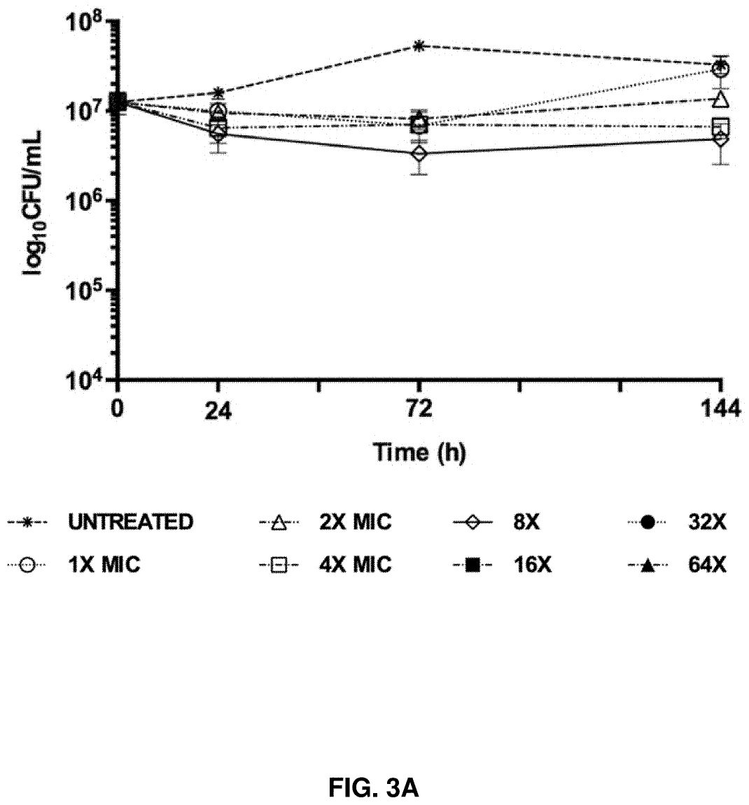

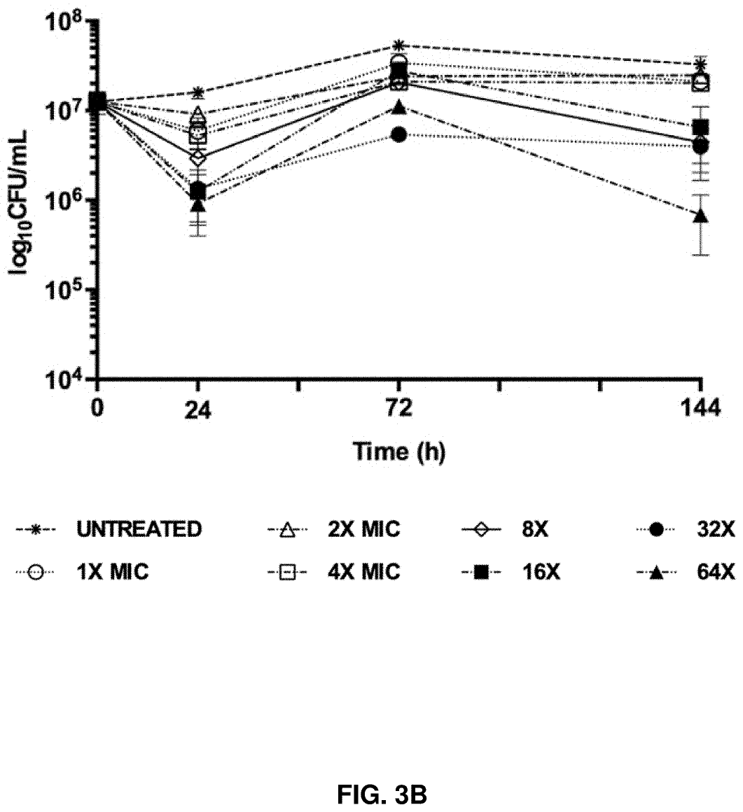

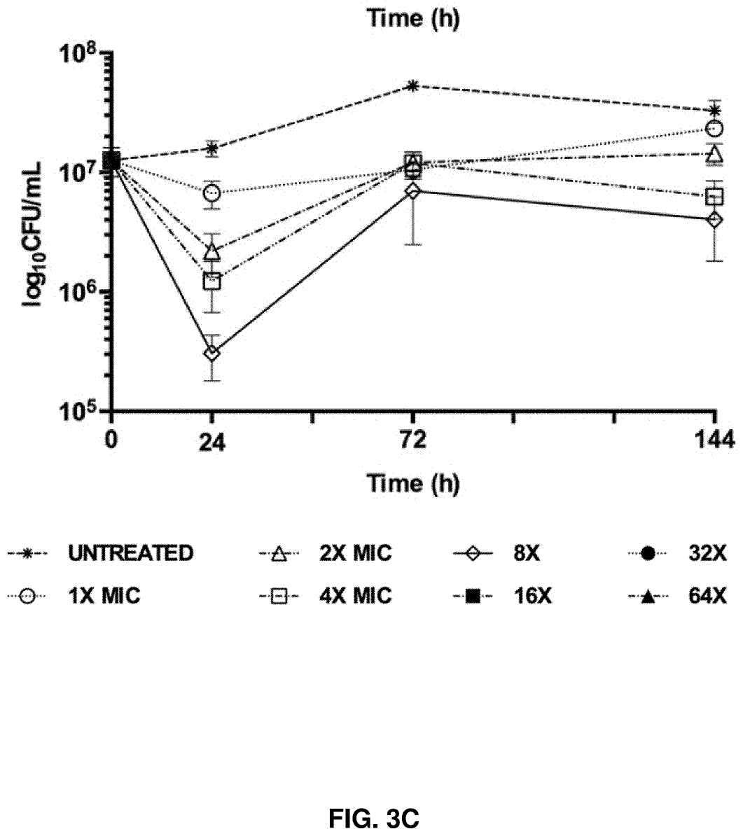

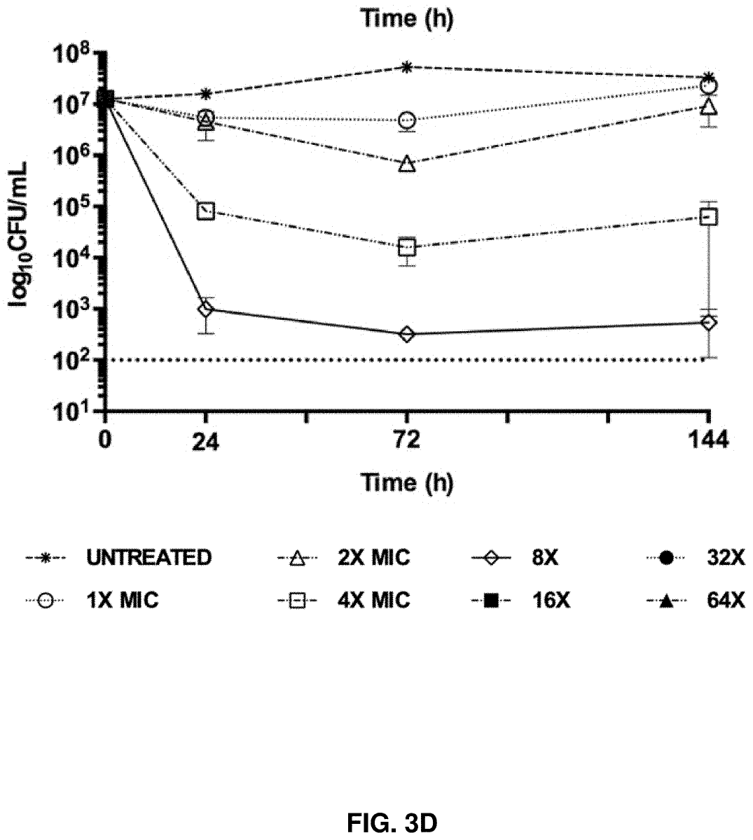

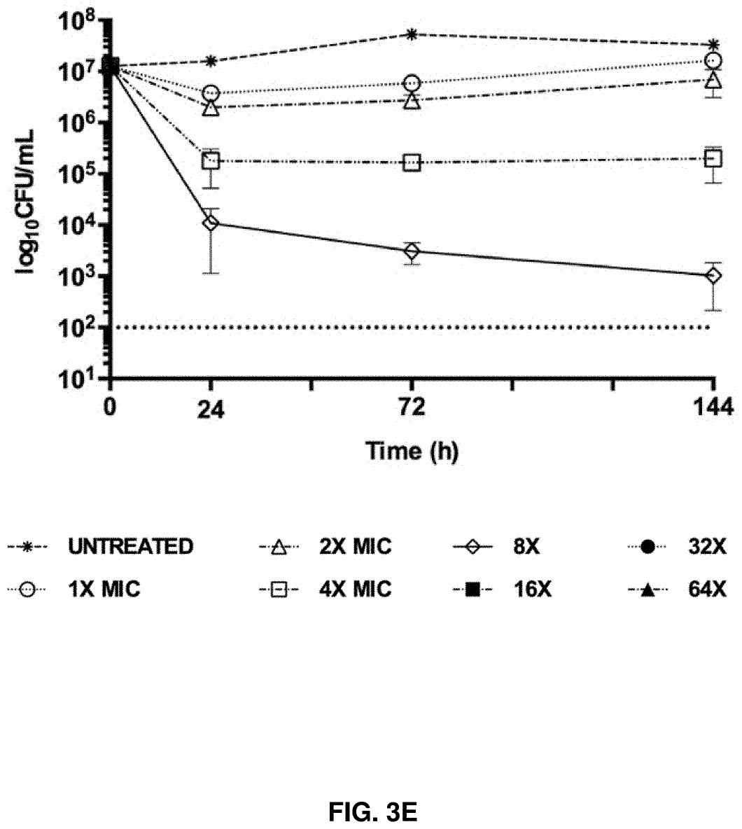

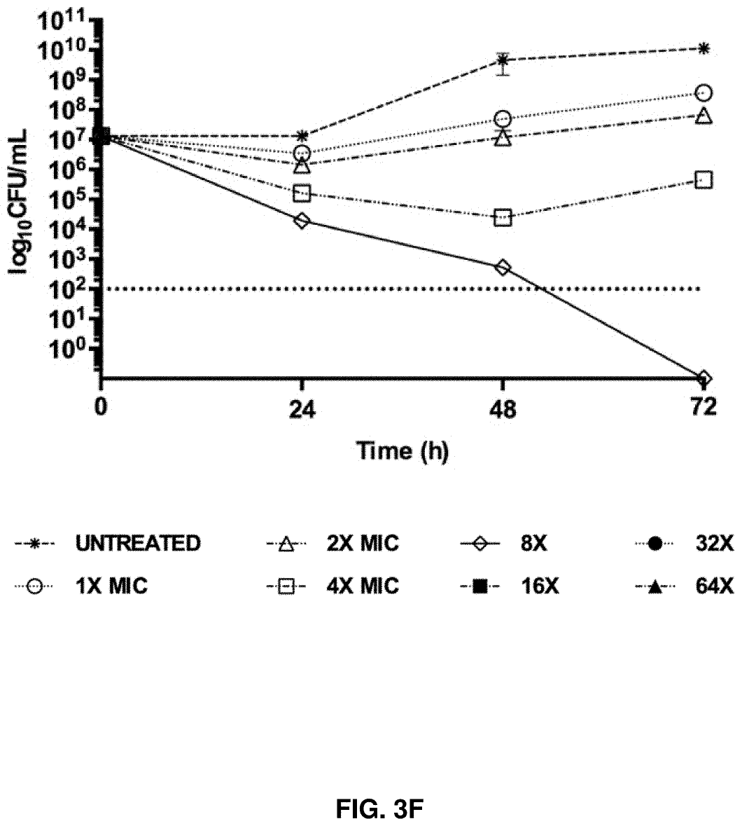

Next, time-kill kinetics experiments were conducted on the five active compounds to validate their activity by CFU enumeration and determine their mode of action. FIG. 3A-FIG. 3F are charts of exemplary of bactericidal activity of gold(III) macrocycles and chelates against Mtb and Mab. Mtb and Mab were grown to log phase and diluted in 7H9 OADC. Mtb was treated with all compounds for up to 6 days (FIG. 3A to FIG. 3E), and Mab with only compound 14 for up to 3 days (FIG. 3F). Compound concentrations ranged from MIC to 8-fold MIC for 8 (FIG. 3A), 11 (FIG. 3C), 14 (FIG. 3D—Mtb & FIG. 3F—Mab) and 15 (FIG. 3E); and up to 64-fold MIC for 10 (FIG. 3B). After each time point (Mtb—0, 24, 72 and 144 h; Mab—0, 24, 48 and 72 h) samples were taken from treated and untreated wells and plated on 7H10 OADC for colony enumeration. As seen in FIG. 3A and B, 8 and 10 inhibited Mtb by less than 1-log CFU/mL at 1, 2, 4 and 8-fold MIC. Even though 10 had the lowest MIC, only moderate bacterial killing (1-log decrease relative to vehicle control) was observed at 64-fold MIC. Considering the latest experimental time-point (144 h), 11 was bacteriostatic at 8, 4 and 2-fold MIC, but not at MIC, suggesting that inhibition of the luminescent signal occurred which could have skewed the MIC obtained for this compound using Mtb-Lux. Nonetheless, a >1-log decrease in CFU/mL was noted with this compound at an early time-point (24 h) which suggests that some bactericidal activity is present, and disappears in later time-points (FIG. 3C).

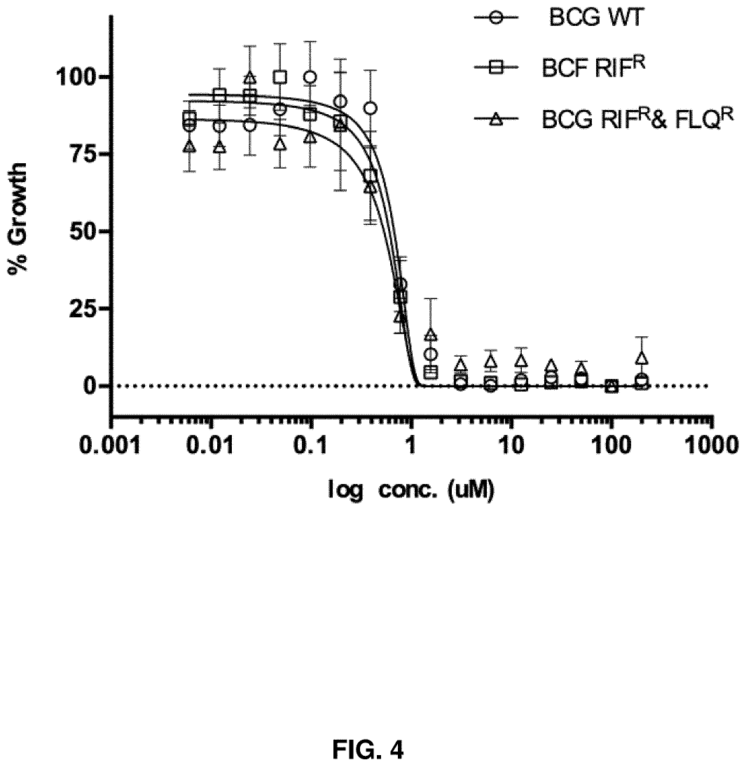

Two of the gold(III) chelates, 14 and 15, exhibited rapid and potent bactericidal activity. Treatment with concentrations at 4- and 8-fold of MIC resulted in ˜2-4-log decreases in CFU within 24 h. However, 14 was more potent based on the magnitude of the decrease in CFU/mL. Additionally, a slight decrease in CFU/mL (<1 log) was observed at 24 h upon treatment with 1 and 2-fold MIC of 14 and 15 followed by recovery to vehicle control levels at the later time-points. FIG. 4 is a chart of an example showing that drug resistant M. bovis BCG is sensitive to compound 14, according to various embodiments. Strains of M. bovis BCG included: wild type (BCG WT), a RIF resistant strains with rpoB mutation (BCG RIFR), a RIF and FLQ resistant triple mutant strain containing mutations in gyrA, gyrB and rpoB (BCG RIF^{R} & FLQ^{R}). Bacterial cultures were grown to log phase, diluted in 7H9 OADC and treated for 5 days after which the luminescence was read. Concentrations of compounds ranged from 200 μM to 0.006 μM in 2-fold 16-point serial dilutions.

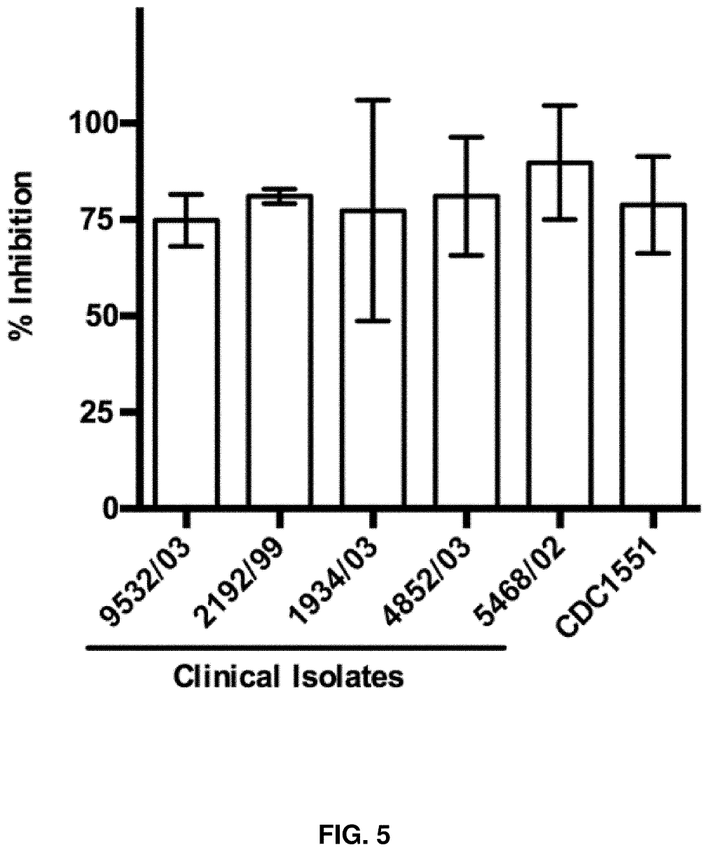

Given the potent bactericidal activity of 14 against Mtb, it was determined if the mode of action was similar against Mab. Importantly, the synthesis of this molecule provided high yields enabling further examination of its activity. The mode of action of 14 was evaluated against Mab at 1,2,4 and 8-fold MIC (FIG. 5). FIG. 5 is a chart of an example showing that compound 14 is active against clinical isolates of Mtb, according to various embodiments. Five clinical isolates of Mtb from different phylogenetic lineages (Table 1) and the reference strain CDC1551 were treated with 5 μM of compound 14 for 5 days. Resazurin was added after treatment and plates were incubated over night after which the fluorescence was read. Percent inhibition values are calculated relative to DMSO and RIF controls.

Since Mab is a fast-growing mycobacterium, the experiment was carried out for 72 h instead of 144 h, and data collection was done every 24 h. Similar to the observations with Mtb, both 4- and 8-fold MIC yielded significant reductions in CFU. While a 1-log rebound in CFU at later time-points with 4-fold MIC treatment was seen, 8-fold MIC resulted in a 4-log decrease by 48 h with apparent sterilization of the culture by 72 h. These data demonstrate the highly bactericidal activity of 14 at this concentration against Mab.

Compound 14 Shows No Cross-Resistance with Clinically Relevant Drugs

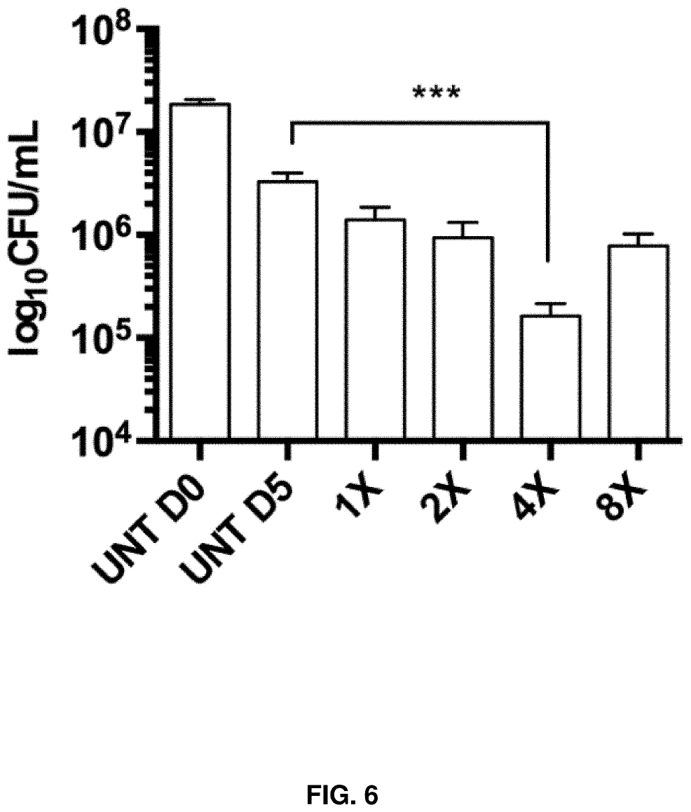

Compound 14 was further characterized in this study considering its potent bactericidal activity profile against both Mtb and Mab. Gold(III) macrocyles with similar scaffolds have been shown to target mammalian topoisomerase 1B [32]. Since fluoroquinolones (FLQ) target a bacterial topoisomerase family of enzymes (DNA gyrase, a type II topoisomerase) [33], it was sought to determine if FLQR mycobacteria would be cross resistant to 14. Additionally, cross-resistance with RIF was investigated due the high incidence of RIFR Mtb strains in the clinic [34]. Two drug-resistant M. bovis BCG strains were developed: i) BCG RIFR & FLQR with mutations in gyrA (specify the mutation), gyrB (specify the mutation) and rpoB (specify the mutation); ii) BCG RIF^{R} with a mutation in rpoB (specify mutation). The mutations were confirmed by sequencing (Table 1). Further validation was done by determining the MICs of ciprofloxacin (CIP), moxifloxacin (MOX) and RIF against these strains and the parent wild type strain (BCG WT). The MIC of CIP, MOX and RIF against BCG FLQ^{R} & RIF^{R} was higher than their MIC against WT. The MIC of RIF against BCG RIF^{R} was the same as the MIC against BCG RIF^{R} & FLQ^{R} (FIG. 51). When 14 was tested against these strains all the MICs were equal to BCG WT suggesting that there is no cross-resistance to 14 in FLQ^{R} and RIF^{R} strains (FIG. 6). FIG. 6 is a chart of an example showing that compound 14 is active against dormant Mtb, according to various embodiments. Cultures of Mtb adapted for 9 days in multiple stress conditions were treated for 5 days with 14 at 1, 2, 4 and 8-fold MIC. After treatment samples were taken for plating on 7H10 agar media. Colonies were counted after 3 weeks of incubation.

### Compound 14 is Active Against Clinical Isolates and Dormant Mtb

To further establish the potential clinical utility of this compound, the activity of 14 was tested against 1) a panel of 5 clinical isolates of Mtb from different phylogenetic lineages (Table 1) and 2) dormant, phenotypically drug-tolerant Mtb.

Dormant Mtb is highly tolerant to front-line TB drugs, severely delaying bacterial clearance during treatment [35]. In this study, the activity of 14 was investigated against non-replicating bacilli pre-adapted in a combination of dormancy-inducing conditions (acidic pH, hypoxia and nutrient starvation). A >1-log decrease in CFU/mL was observed for dormant Mtb cultures treated with 4-fold MIC of 14 for 5 days as compared to the vehicle controls, suggesting killing of phenotypically tolerant Mtb by 14.

### Compound 14 Inhibits Bacterial Topisomerase 1A

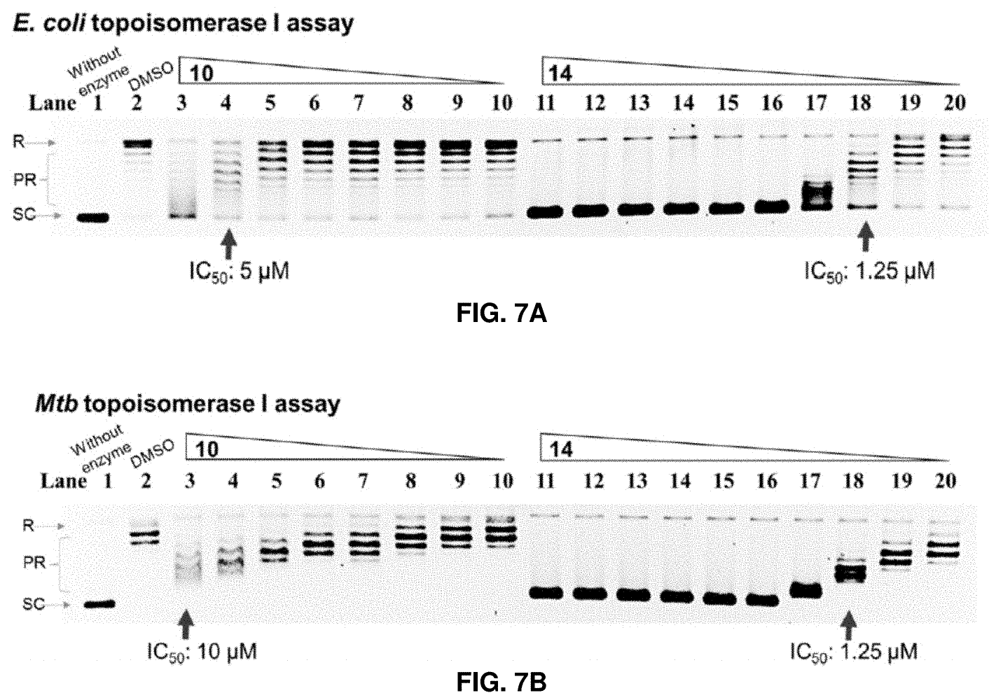

FIG. 7A is an example assay carried out with 10 ng EcTopo1, illustrating inhibition of bacterial topoisomerase 1A relaxation activity by compound 14, in which lane 1 is a control reaction without enzyme added; lane 2, DMSO control; lanes 3 to 10 are reactions with compound 10 at concentrations of 10, 5, 2.5, 1.25, 0.63, 0.31, 0.15, and 0.075 μM, respectively; and lanes 11 to 20 are reactions with compound 14 at concentrations of 160, 80, 40, 20, 10, 5, 2.5, 1.25, 0.62, and 0.31 μM, respectively.

FIG. 7B is an example assay carried out with 25 ng MtbTopo1, illustrating inhibition of bacterial topoisomerase 1A relaxation activity by compound 14, in which lane 1 is a control reaction without enzyme added; lane 2, DMSO control; lanes 3 to 10 are reactions with compound 10 at concentrations of 10, 5, 2.5, 1.25, 0.63, 0.31, 0.15, and 0.075 μM, respectively; and lanes 11 to 20 are reactions with compound 14 at concentrations of 160, 80, 40, 20, 10, 5, 2.5, 1.25, 0.62, and 0.31 μM, respectively.

In FIGS. 7A and 7B, the IC₅₀ was determined as the drug concentration which inhibited 50% of the relaxation ability. In FIG. 7A and 7B, R indicates relaxed circular DNA; PR indicates partially relaxed DNA; and SC indicates supercoiled DNA. As seen in FIG. 7A and FIG. 7B, at 5 μM 14 was similarly active against all 5 Mtb clinical isolates and Mtb CDC1551.

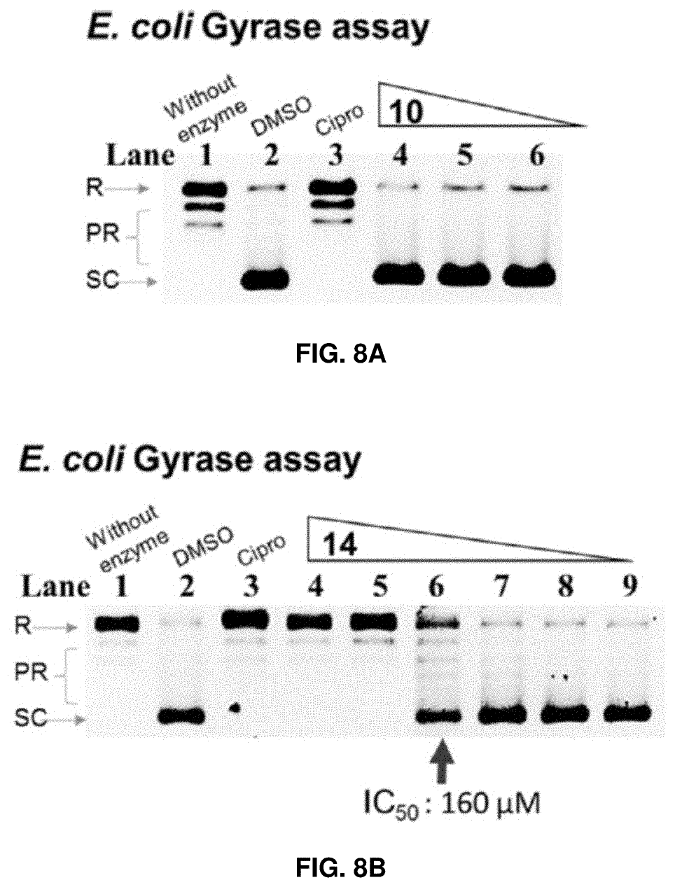

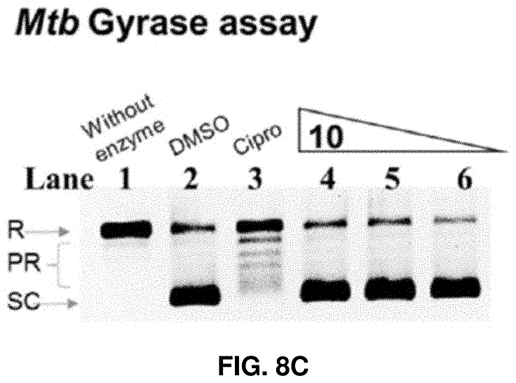

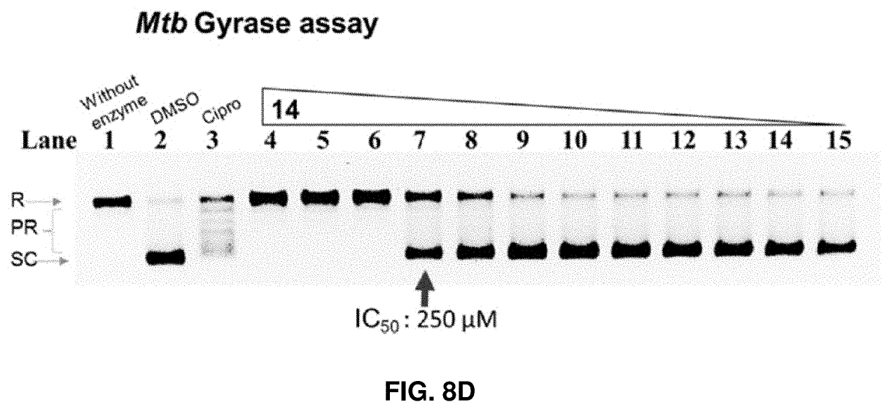

FIG. 8A is an example assay of E. coli gyrase activity in the presence of compound 10, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 6 being reactions with compound 10 at concentrations of 10, 5, and 2.5 μM, respectively, according to various embodiments.

FIG. 8B is an example assay of E. coli gyrase activity in the presence of compound 14, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 9 being reactions with compound 14 at concentrations of 500, 250, 160, 80, 40, and 20 μM, respectively according to various embodiments. FIG. 8C is an example assay of M. tuberculosis gyrase activity in the presence of compound 10, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lanes 4 to 6, reactions with compound 10 at concentrations of 10, 5, and 2.5 μM, respectively according to various embodiments.

FIG. 8D is an example assay of M. tuberculosis gyrase activity in the presence of compound 14, illustrating inhibition of bacterial gyrase supercoiling activity by compound 14, with lane 1 being a control reaction without enzyme added, lane 2 being a DMSO control, lane 3 being a positive-control reaction with 150 μM ciprofloxacin (Cipro), and lane 4 to 15, reactions with compound 14 at concentrations of 1,000, 750, 500, 250, 160, 80, 40, 20, 10, 5, 2.5, and 1.25 μM, respectively, according to various embodiments.

In FIG. 8A-FIG. 8D, the IC₅₀ was determined as the drug concentration which inhibited 50% of the supercoiling activity. In FIG. 8A-FIG. 8D, R indicates relaxed circular DNA; PR indicates partially relaxed DNA; and SC indicates supercoiled DNA. In contrast, RIF at 4-fold MIC did not kill dormant bacilli under these same conditions [26], highlighting the potential of 14 in a more clinically relevant context.

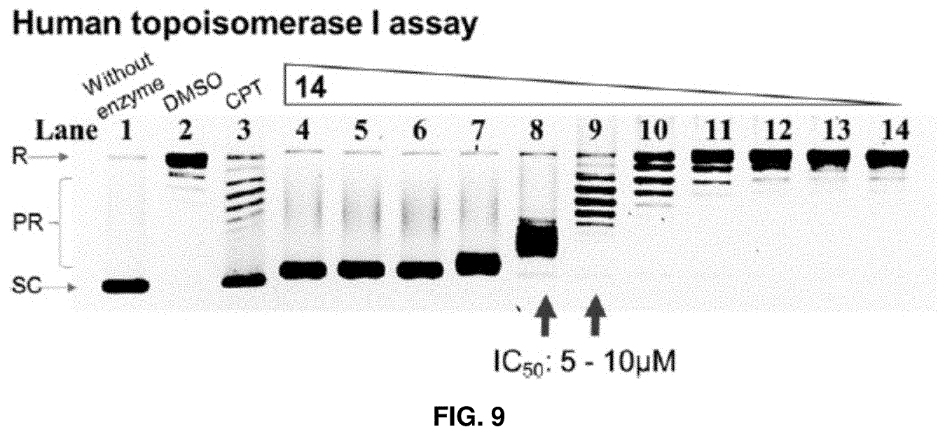

FIG. 9 is an example assay illustrating human topoisomerase 1B (hTopo1) relaxation activity by compound 14, in which lane 1 is a control reaction without enzyme added; lane 2 is a DMSO control; lane 3 is a positive-control reaction with 200 μM camptothecin (CPT); and lanes 4 to 14 are reactions with compound 14 at concentrations of 160, 80, 40, 20, 10, 5, 2.5, 1.25, 0.62, 0.31, and 0.15 μM, respectively. In FIG. 9, the IC₅₀ was determined as the drug concentration which inhibited 50% of the relaxation activity. R indicates relaxed circular DNA; PR indicates partially relaxed DNA; and SC indicates supercoiled DNA.

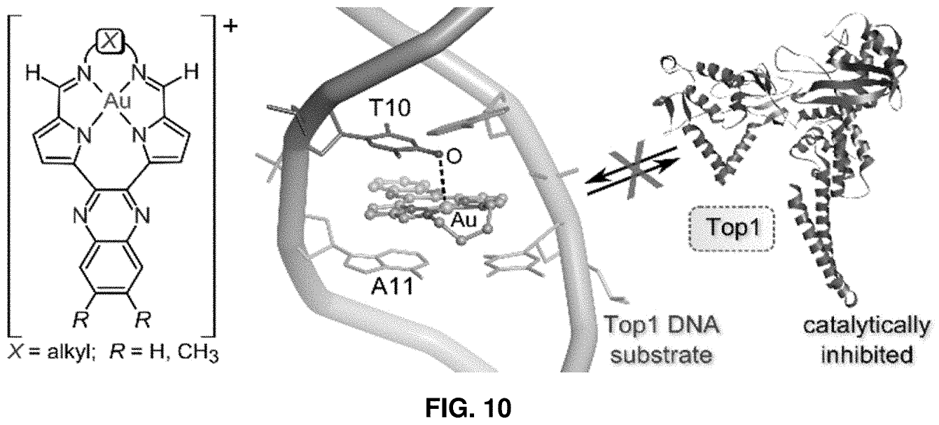

FIG. 10 is a schematic diagrammatic model of an example, illustrating topoisomerase inhibition by gold macrocycles, according to various embodiments.

### Discussion

DNA topoisomerases are ubiquitous among living organisms. These enzymes, which are divided in two major classes (type I and type II) depending on their mode of action, provide the essential function of modulating DNA topology [36]. For this reason, they are often used as targets of therapeutic agents against human disease [37]. Recently, a class of gold(III) macrocycles was synthesized with moderate cytotoxic activity targeting human topoisomerase type I [32]. Type I enzymes are usually conserved across the different domains of life not only in function, but also amino acid sequence at the active site [38]. Considering this, and the essentiality of mycobacterial topo I [39-41], it was sought to examine the activity of this novel class of gold complexed compounds against two important mycobacterial pathogens—Mtb and Mab.

Metal complexed compounds have been historically used against bacterial infections. Different types of metal-complexed antimicrobials can display a wide range of mechanisms, including increased ROS production, depletion of antioxidants, disruption of protein, membrane function and nutrient assimilation [42]. Recently, the FDA approved gold compound auranofin, a gold(I) complexed scaffold, was tested against several pathogens including Mtb. Even though this compound was bactericidal against Mtb, this activity was broad-spectrum [43], which is considered incompatible with TB treatment given the long combination therapy required to cure the disease. Gold(I) compounds have been shown to have a high affinity for protein ligands, can inhibit biofilm formation and cause cell wall damage in gram-negative bacteria [44, 45]. Gold(III) complexes react with RNA, and zinc-finger PARP domains as well as topoisomerases [32, 46].

Mycobacterial diseases pose a major hurdle in global health mainly because of an increased difficulty to clear these infections with antibiotics. In the search for effective anti-mycobacterials , gyrase (type(II) topoisomerase) inhibitors such as fluoroquinolones have become well-established tools for combating drug-resistant Mtb, and evidence suggests they could shorten TB treatment duration in combination therapy [47, 48]. Although Mtb topo I inhibitors are not as widely studied as gyrase inhibitors, recent studies screening small-molecules against Mtb have uncovered promising hit compounds targeting this class of enzymes [49, 50]. However, the study provided herein takes a reverse approach by investigating whether human topo I inhibitors can be good candidates for anti-mycobacterials. This notion that eukaryotic topoisomerase inhibitors could cross-over to the anti-mycobacterial drug discovery pipeline has been previously introduced [51], though concerns regarding selectivity can be a pitfall of this approach. Nonetheless, the gold complexes in the study were active against Mtb and Mab with Sls (selectivity indices) far beyond 10 for Mtb compared to J774 IC₅₀, which is early evidence of the selective nature of these compounds.

In the data provided herein, identified and characterized is the activity of 5 gold(III) macrocycles against Mtb. Using time-to-kill curves as well as MIC provided meaningful information regarding the modes of action of each structure. These inhibitors included both interfacial poisons, locking the DNA-enzyme complex in the nicked state, and catalytic inhibitors which directly block the enzyme's activity [52]. The data suggests 14 not only had a low MIC of 0.98 μM, but is also highly bactericidal. Also, killing of actively replicating bacilli by 14 occurs early-on, however bacteria are not fully cleared by these compounds. Conversely, this kinetic profile could be due to compound instability over time, lowering the level of active compound present in the culture. Even though 10 had the lowest MIC (0.12 μM), this molecule was almost completely bacteriostatic against Mtb. Nonetheless, bacteriostatic drugs remain useful in the treatment of TB and other mycobacterial diseases. Interestingly the Au(III) ion from 10 cannot be lost since it is very tightly bound. On the other hand, reductive demetallation of 14 by glutathione (GSH) has been observed (rate constant, k=0.0463±0.0002 M⁻¹ s⁻¹ at physiological pH and temperature). This demetallation leads to the formation of a free bis(pyrrole) ligand which could account for the potent bactericidal activity of 14 as compared to 10. Moreover, both 10 and 14 were able to inhibit topoisomerase I and neither had significant activity against topoisomerase II. Investigating the activity of the bis(pyrrole) ligand of 14, and the role of mycothiol (mycobacterial GSH) for the effectiveness of 14 will help clarify the compound's mechanism of action.

The incidence of MDR and XDR Mtb strains is rising worldwide [53]. The long treatment time required to fully eradicate dormant bacilli weakens patient compliance leading to a cycle that can only be broken by more effective drugs against resistant and phenotypically tolerant bacteria. Since 14 had the most promising activity, it was also studied in these clinically relevant contexts of TB. RIF and fluoroquinolone-resistant strains of BCG and WT BCG were equally inhibited by this compound; as were clinical isolates when compared to the Mtb reference strain CDC1551. Furthermore, an almost 2-log decrease in CFU/mL was observed in dormant Mtb treated with 14. Absence of cross-resistance from fluoroquinolone-resistant strains to 14 implies a novel mechanism of action for this compound. As discussed, this class of compounds was found to target human type I topoisomerase (but had unexpectedly low cytotoxicity in hollow fiber assays in mice). The newly identified target for bactericidal gold(III) chelate 14 in this disclosure is bacterial topoisomerase 1A, an essential enzyme for bacterial growth, survival, and cell division. Together these findings highlight the potential for this class of compounds to be developed into a novel treatment option for TB.

Mab is known to cause chronic lung, skin and soft tissue infections. Since it is resistant to front-line TB drugs, treatment options usually remain limited to macrolides and aminoglycosides and a beta-lactam antibiotic [54]. Drugs such as amikacin and clarithromycin used in the clinic are very ineffective against Mab, consistent with poor bactericidal activity that was previously observed in vitro even at 32 and 64-fold MIC [56], and the relapse rates after treatment are alarmingly high [57, 58]. The findings in the present study suggest metal complexes could provide effective alternative treatment for fast-growing mycobacterial infections. Previous reports of metal complexes active against mycobacteria further supports this notion. For instance, the activity sulfonamide metal complexes was recently demonstrated against fast-growing mycobacteria [55]. The results herein show that gold(III) complexes can potently inhibit Mab, with MICs as low as 5.4 μM for 10. Additionally, 14 was highly bactericidal against these bacteria at 8-fold MIC and moderate killing was observed at half that concentration.

In this study, uncovered was the potential of gold(III) macrocycles and chelates, a novel class of compounds, as antimicrobial agents against important mycobacterial pathogens. One key target of these compounds has been confirmed in this work, namely bacterial topoisomerase 1A. which is valuable in the characterization of their mechanism of action as well as optimization for more selective inhibition of the mycobacterial enzyme through structure-activity relationship studies. Since these gold(III) complexes differ greatly in their mode of action, future work will also focus on determining the aspects of the scaffolds that provide bactericidal versus bacteriostatic activity. Moreover, preliminary work testing the tolerability of mice to 10 shows this compound was tolerated at 400 mg/kg in this in vivo model. This is encouraging evidence for future work with active compounds in this class using in vivo models of Mtb and Mab infection. In conclusion, characterized herein are optimal drug candidates for hit-to-lead development which can ultimately lead to more effective treatment options for mycobacterial infections.

### REFERENCES

1. Gold, B. and C. Nathan, Targeting Phenotypically Tolerant Mycobacterium tuberculosis. Microbiology Spectrum, 2017. 5(1).
2. Bar-On, O., et al., Increasing nontuberculous mycobacteria infection in cystic fibrosis. Journal of Cystic Fibrosis, 2015. 14(1): p. 53-62.
3. Choi, G. E., et al., Macrolide treatment for Mycobacterium abscessus and Mycobacterium massiliense infection and inducible resistance. Am J Respir Crit Care Med, 2012. 186(9): p. 917-25.
4. Maurer, F. P., et al., Erm(41)-dependent inducible resistance to azithromycin and clarithromycin in clinical isolates of Mycobacterium abscessus. Journal of Antimicrobial Chemotherapy, 2014. 69(6): p. 1559-1563.
5. Lee, M. R., et al., Mycobacterium abscessus Complex Infections in Humans. Emerg Infect Dis, 2015. 21(9): p. 1638-46.
6. Hoagland, D. T., et al., New agents for the treatment of drug-resistant Mycobacterium tuberculosis. Adv Drug Deliv Rev, 2016. 102: p. 55-72.
7. Berners-Price, S. J. and A. Filipovska, Gold compounds as therapeutic agents for human diseases. Metallomics, 2011. 3(9): p. 863-873.
8. Kelland, L., The resurgence of platinum-based cancer chemotherapy. Nat Rev Cancer, 2007. 7(8): p. 573-84.
9. Finkelstein, A. E., et al., Auranofin—New Oral Gold Compound for Treatment of Rheumatoid-Arthritis. Annals of the Rheumatic Diseases, 1976. 35(3): p. 251-257.
10. Sutton, B. M., et al., Oral Gold—Antiarthritic Properties of Alkylphosphinegold Coordination Complexes. Journal of Medicinal Chemistry, 1972. 15(11): p. 1095-+.
11. Li, X. N., et al., Functional Gold Nanoparticles as Potent Antimicrobial Agents against Multi-Drug-Resistant Bacteria. Acs Nano, 2014. 8(10): p. 10682-10686.
12. Lima, E., et al., Gold nanoparticles as efficient antimicrobial agents for Escherichia coli and Salmonella typhi. Chemistry Central Journal, 2013. 7.
13. Shamaila, S., et al., Gold Nanoparticles: An Efficient Antimicrobial Agent against Enteric Bacterial Human Pathogen. Nanomaterials, 2016. 6(4).
14. Zhou, Y., et al., Antibacterial activities of gold and silver nanoparticles against Escherichia coli and bacillus Calmette-Guerin. Journal of Nanobiotechnology, 2012. 10.
15. Benedek, T. G., The history of gold therapy for tuberculosis. J Hist Med Allied Sci, 2004. 59(1): p. 50-89.
16. Agertt, V. A., et al., Identification of antimicrobial activity among new sulfonamide metal complexes for combating rapidly growing mycobacteria. Biometals, 2016. 29(5): p. 807-816.
17. Barbosa, A. R., et al., Potential of Casiopeinas(R) Copper Complexes and Antituberculosis Drug Combination against Mycobacterium tuberculosis. Chemotherapy, 2016. 61(5): p. 249-55.
18. Koh, W. J., et al., Pulmonary TB and NTM lung disease: comparison of characteristics in patients with AFB smear-positive sputum. Int J Tuberc Lung Dis, 2006. 10(9): p. 1001-7.
19. Nessar, R., et al., Mycobacterium abscessus: a new antibiotic nightmare. Journal of Antimicrobial Chemotherapy, 2012. 67(4): p. 810-818.
20. Abraham, E., ATS/IDSA statement: Diagnosis, treatment, and prevention of nontuberculous mycobacterial diseases (vol 175, pg 394, 2007). American Journal of Respiratory and Critical Care Medicine, 2007. 175(7): p. 744-745.
21. Crabol, Y., et al., Rifabutin: where do we stand in 2016? Journal of Antimicrobial Chemotherapy, 2016. 71(7): p. 1759-1771.
22. Dinah Binte Aziz, et al., Rifabutin Is Active Against Mycobacterium abscessus Complex. Antimicrobial agents and Chemotherapy, 2017.
23. Chopra, S., et al., Identification of antimicrobial activity among FDA-approved drugs for combating Mycobacterium abscessus and Mycobacterium chelonae. Journal of Antimicrobial Chemotherapy, 2011. 66(7): p. 1533-1536.
24. Kaushik, A., et al., Carbapenems and Rifampin Exhibit Synergy against Mycobacterium tuberculosis and Mycobacterium abscessus. Antimicrobial Agents and Chemotherapy, 2015. 59(10): p. 6561-6567.
25. Greendyke, R. and T. F. Byrd, Differential antibiotic susceptibility of Mycobacterium abscessus variants in biofilms and macrophages compared to that of planktonic bacteria. Antimicrobial Agents and Chemotherapy, 2008. 52(6): p. 2019-2026.
26. Rodrigues Felix, C., et al., Selective Killing Of Dormant Mycobacterium tuberculosis By Marine Natural Products. Antimicrob Agents Chemother, 2017.
27. Zhang, J. H., T. D. Y. Chung, and K. R. Oldenburg, A simple statistical parameter for use in evaluation and validation of high throughput screening assays. Journal of Biomolecular Screening, 1999. 4(2): p. 67-73.
28. Lambert, R. J. W. and J. Pearson, Susceptibility testing: accurate and reproducible minimum inhibitory concentration (MIC) and non-inhibitory concentration (NIC) values. Journal of Applied Microbiology, 2000. 88(5): p. 784-790.
29. Palomino, J. C., et al., Resazurin microtiter assay plate: Simple and inexpensive method for detection of drug resistance in Mycobacterium tuberculosis. Antimicrobial Agents and Chemotherapy, 2002. 46(8): p. 2720-2722.
30. Deb, C., et al., A Novel In Vitro Multiple-Stress Dormancy Model for Mycobacterium tuberculosis Generates a Lipid-Loaded, Drug-Tolerant, Dormant Pathogen. Plos One, 2009. 4(6).
31. Andreu, N., et al., Optimisation of Bioluminescent Reporters for Use with Mycobacteria. Plos One, 2010. 5(5).
32. Akerman, K. J., et al., Gold(III) macrocycles: nucleotide-specific unconventional catalytic inhibitors of human topoisomerase I. J Am Chem Soc, 2014. 136(15): p. 5670-82.
33. Aldred, K. J., R. J. Kerns, and N. Osheroff, Mechanism of quinolone action and resistance. Biochemistry, 2014. 53(10): p. 1565-74.
34. WHO, Global tuberculosis report. 2016.
35. Gold, B. and C. Nathan, Targeting Phenotypically Tolerant Mycobacterium tuberculosis. Microbiol Spectr, 2017. 5(1).
36. Roca, J., The mechanisms of DNA topoisomerases. Trends Biochem Sci, 1995. 20(4): p. 156-60.
37. Pommier, Y., Drugging topoisomerases: lessons and challenges. ACS Chem Biol, 2013. 8(1): p. 82-95.
38. Baker, N. M., R. Rajan, and A. Mondragon, Structural studies of type I topoisomerases. Nucleic Acids Res, 2009. 37(3): p. 693-701.
39. Sassetti, C. M., D. H. Boyd, and E. J. Rubin, Genes required for mycobacterial growth defined by high density mutagenesis. Mol Microbiol, 2003. 48(1): p. 77-84.
40. Ahmed, W., et al., Conditional silencing of topoisomerase I gene of Mycobacterium tuberculosis validates its essentiality for cell survival. FEMS Microbiol Lett, 2014. 353(2): p. 116-23.
41. Ahmed, W., et al., Reduction in DNA topoisomerase I level affects growth, phenotype and nucleoid architecture of Mycobacterium smegmatis. Microbiology, 2015. 161(Pt 2): p. 341-53.
42. Lemire, J. A., J. J. Harrison, and R. J. Turner, Antimicrobial activity of metals: mechanisms, molecular targets and applications. Nat Rev Microbiol, 2013. 11(6): p. 371-84.
43. Harbut, M. B., et al., Auranofin exerts broad-spectrum bactericidal activities by targeting thiol-redox homeostasis. Proc Natl Acad Sci USA, 2015. 112(14): p. 4453-8.
44. Shaw, I. C., Gold-based therapeutic agents. Chem Rev, 1999. 99(9): p. 2589-600.
45. Glisic, B. D. and M. I. Djuran, Gold complexes as antimicrobial agents: an overview of different biological activities in relation to the oxidation state of the gold ion and the ligand structure. Dalton Trans, 2014. 43(16): p. 5950-69.
46. Glisic, B. D., U. Rychlewska, and M. I. Djuran, Reactions and structural characterization of gold(III) complexes with amino acids, peptides and proteins. Dalton Trans, 2012. 41(23): p. 6887-901.
47. D'Ambrosio, L., et al., New anti-tuberculosis drugs and regimens: 2015 update. ERJ Open Res, 2015. 1(1).
48. Burman, W. J., et al., Moxifloxacin versus ethambutol in the first 2 months of treatment for pulmonary tuberculosis. Am J Respir Crit Care Med, 2006. 174(3): p. 331-8.
49. Sandhaus, S., et al., Small-Molecule Inhibitors Targeting Topoisomerase I as Novel Antituberculosis Agents. Antimicrob Agents Chemother, 2016. 60(7): p. 4028-36.
50. Godbole, A. A., et al., Targeting Mycobacterium tuberculosis topoisomerase I by small-molecule inhibitors. Antimicrob Agents Chemother, 2015. 59(3): p. 1549-57.
51. Godbole, A. A., et al., Inhibition of Mycobacterium tuberculosis topoisomerase I by m-AMSA, a eukaryotic type II topoisomerase poison. Biochem Biophys Res Commun, 2014. 446(4): p. 916-20.
52. Denny, W. A., Dual topoisomerase I/II poisons as anticancer drugs. Expert Opin Investig Drugs, 1997. 6(12): p. 1845-51.
53. Koul, A., et al., The challenge of new drug discovery for tuberculosis. Nature, 2011. 469(7331): p. 483-90.
54. Nessar, R., et al., Mycobacterium abscessus: a new antibiotic nightmare. J Antimicrob Chemother, 2012. 67(4): p. 810-8.
55. Agertt, V. A., et al., Identification of antimicrobial activity among new sulfonamide metal complexes for combating rapidly growing mycobacteria. Biometals, 2016. 29(5): p. 807-16.
56. Ferro, B. E., et al., Time-kill kinetics of antibiotics active against rapidly growing mycobacteria. J Antimicrob Chemother, 2015. 70(3): p. 811-7.
57. Ferro, B. E., et al., Failure of the Amikacin, Cefoxitin, and Clarithromycin Combination Regimen for Treating Pulmonary Mycobacterium abscessus Infection. Antimicrob Agents Chemother, 2016. 60(10): p. 6374-6.
58. Lerat, I., et al., In vivo evaluation of antibiotic activity against Mycobacterium abscessus. J Infect Dis, 2014. 209(6): p. 905-12.

Work on this invention was supported by grants from the Cystic Fibrosis Foundation under award number ROHDE15G0.

What is claimed is:

1. A method of treating an object infected with microbes or at risk of infection comprising administering an effective amount of a composition comprising a gold macrocycle or chelate compound or a pharmaceutically acceptable salt thereof, and optionally a pharmaceutically acceptable carrier tothe object.

2. The method of claim 1, wherein the object is a living subject infected with microbes or at risk of infection.

3. The method of claim 2, wherein the living subject is a human.

4. The method of claim 1, wherein the microbes comprise Mycobacterium spp.

5. The method of claim 4, wherein the Mycobacterium spp comprises M. tuberculosis or M. abscessus, or both.

6. A method of treating a subject infected with a Mycobacterium spp., the method comprising administering a therapeutically effective amount of a composition comprising one or more gold macrocycle and/or chelate compounds.

7. The method of claim 6, wherein the one or more gold macrocycle and/or chelate compounds comprises a gold(III) complex of compound 8, 10, 11, 14 or 15, or a pharmaceutically acceptable salt thereof.

8. The method of claim 7, wherein the composition comprises compound 14.

9. The method of claim 1, wherein the object is an inanimate object having a surface, wherein the composition is formulated for application to the surface infected or at risk of infection by mycobacterium.

10. The method of claim 1, wherein the effective amount is an amount that delivers 0.1 to 100 μM to the Mycobacterium spp.

11. The method of claim 2, wherein the composition comprises gold complex compound 8, 10, 11, 14 or 15, or a pharmaceutically acceptable salt thereof, or a combination thereof.

12. The method of claim 7, wherein the pharmaceutically acceptable salt thereof comprises hexofluorophosphate or chloride.

13. The method of claim 1, further comprising co-administering an adjunct antibacterial compound.

14. The method of claim 6, further comprising co-administering an adjunct antibacterial compound.
